(19) United States
(12) Patent Application Publication
(10) Pub. No.: US 2022/0008124 A1
(43) Pub. Date: Jan. 13, 2022

(54) TWO-IN-ONE CATHETER FOR REAL-TIME ULTRASOUND MONITORING AND RADIOFREQUENCY ABLATION

(71) Applicant: ZHEJIANG UNIVERSITY, Hangzhou City, Zhejiang Province (CN)

(72) Inventors: Liangrong ZHENG, Hangzhou City, Zhejiang Province (CN); Zewei SUN, Hangzhou City, Zhejiang Province (CN)

(21) Appl. No.: 16/965,009

(22) PCT Filed: Jul. 7, 2019
(86) PCT No.: PCT/CN2019/094962

§ 371 Date: Jul. 26, 2020

(30) Foreign Application Priority Data
Apr. 1, 2019 (CN) 201910257969.X

(51) Int. Cl. A61B 18/14; A61B 8/12

(52) U.S. Cl. CPC A61B 18/1492; A61B 2018/00148; A61B 8/12

(57) ABSTRACT

Provided is a two-in-one catheter for real-time ultrasound monitoring and radiofrequency ablation, composed of a tube body, an ultrasound probe, cold saline infusion holes, recording electrodes and an ablation electrode. The ablation electrode is a metal mesh or a metal column made of a material with pores. A diameter of a metal wire forming the metal mesh or the metal column or a diameter of an aperture formed by the metal mesh or the metal column is adapted to the same order of magnitude of the clinically common ultrasound wavelength, so that the ultrasound wave can reach the back of the ablation electrode by diffraction for imaging. The ultrasound monitors the entire ablation process. The present disclosure achieves ablation under real-time ultrasound monitoring, ensuring that a predetermined tissue damage range is reached. The two-in-one catheter avoids an unnecessary exchange of catheters during operation, which is time-saving.

### TECHNICAL FIELD

The present application belongs to the field of medical equipment, and relates to a two-in-one catheter for real-time ultrasound monitoring and radiofrequency ablation, and an application of the catheter in radiofrequency ablation.

### BACKGROUND

Radiofrequency ablation technology is widely used in a variety of medical procedures, especially radiofrequency ablation treatment for arrhythmia. The radiofrequency ablation necessarily causes a certain range of tissue damage to ensure an ablation effect. At present, when performing the radiofrequency ablation, since the ablation catheter and the ultrasound catheter are separated, an ablation catheter has to be withdrawn and replaced with an ultrasound catheter to determine the range of tissue damage by ultrasound. This is time-consuming. Further, since the diameter of the ablation electrode is only several millimeters and the tissue damage range is small, it is usually difficult to find the previous ablation point with the ultrasound catheter after the catheter is exchanged. In this regard, it is unlikely to observe the tissue damage situation under ultrasound. Besides, if it is found under ultrasound that the tissue damage range is insufficient, the catheter has to be changed again and it is very troublesome. Therefore, it is necessary to develop an ultrasound and radiofrequency ablation two-in-one catheter, which can save the time for exchanging the catheters and reduce the difficulty of finding the ablation site after the catheters are exchanged, while monitoring the ablation effect in real time using an ultrasound imaging to reach a predetermined tissue damage range, thereby increasing the success rate of surgery and reduce complications.

### SUMMARY

A purpose of the present disclosure is to provide a two-in-one catheter for real-time ultrasound monitoring and radiofrequency ablation, which is composed of a tube body, an ultrasound probe, cold saline infusion holes, recording electrodes and an ablation electrode. The ultrasound probe is located in a tube cavity of the tube body. The ablation electrode is located at a head end of the tube body. The cold saline infusion holes and the recording electrodes are located at a front segment of the tube body. The ablation electrode is a metal mesh or a metal column, and made of a material with pores, in which a diameter of a metal wire used or a diameter of an aperture formed by the metal mesh or the metal column is adapted to the same order of magnitude of the ultrasound wavelengths commonly used in clinical, ranging from 0.05 mm to 0.3 mm. At present, an intraluminal ultrasound frequency commonly used in clinical practice is 5-15M, so that the ultrasound wave can reach the back of the ablation electrode through diffraction for imaging. Thus, the entire ablation process can be monitored by the ultrasound. The cold saline infusion holes have a pore-like structure and includes 6 cold saline infusion holes provided around the tube body, which are configured to infuse cold saline during ablation to prevent formation of eschar at a contact interface between the catheter and the tissue, thereby reducing resistance. The recording electrodes are a pair of ring electrodes made of a platinum material and fixed on a surface of the tube. The recording electrodes are configured to record an electrocardiogram of a myocardial tissue in contact with the catheter, so as to find an ectopic pacemaker of arrhythmia.

The ablation electrode is preferably a metal mesh, and may be made of any metal such as platinum, titanium, copper, iron, stainless steel, etc., and preferably made of platinum or titanium.

The application of the present disclosure includes steps of turning on ultrasound, performing ablation under ultrasound monitoring, and turning off a radiofrequency ablation current when a tissue damage reaches a predetermined range.

The catheter designed according to the present disclosure integrates ultrasound and radiofrequency ablation. The ablation electrode is designed as, for example, a metal mesh or a metal column, which has pores. A diameter of the pores is adapted to the same order of magnitude of a wavelength of the ultrasound wave, so that the ultrasound can reach the back of the ablation electrode by diffraction for imaging. When performing the radiofrequency ablation according to the present disclosure, a real-time monitoring can be performed with the ultrasound without exchanging catheters, which is time-saving and avoids the trouble of finding an ablation site after the catheters are exchanged. The radiofrequency ablation under real-time ultrasound monitoring can also help the operator accurately reach the predetermined tissue damage range, and thus a success rate of surgery can be increased and surgical complications can be recued, which is beneficial to the radiofrequency ablation surgery. The present application is reasonably designed, the preparation and operation thereof are simple and convenient, and thus has the prospects of application.

### BRIEF DESCRIPTION OF DRAWINGS

FIG. 1 is a schematic structural diagram of a ultrasound and radio frequency ablation two-in-one catheter, where 1 denotes a tube body of the catheter, 2 denotes an ultrasound probe located in a tube cavity of the catheter, 3 denotes a cold saline infusion hole, 4 denotes a recording electrode, and 5 denotes an ablation electrode made of a metal mesh or a metal column.

FIG. 2 is images after an ultrasound probe is wrapped with a metal mesh. As shown in the figure, a quality of ultrasound images are not significantly affected by the metal mesh wrapped the ultrasound probe.

FIG. 3 is images of results of radiofrequency ablation using a metal mesh as an ablation electrode. As shown in the figure, the radiofrequency ablation using a metal mesh does not affect the ablation effect and tissue damage can be formed.

FIG. 4 is images of results of ablation when a metal mesh wraps an ultrasound probe and another metal mesh is used as an ablation electrode. As shown in the figure, ultrasound imaging is not significantly affected when the ultrasound probe is wrapped with the metal mesh, and tissue damage can be observed after the ablation with another metal mesh.

FIG. 5 is a diagram of a specific application using the ultrasound and radio frequency ablation two-in-one catheter according to the present disclosure. As shown in the figure, during the use, the recording electrodes 4 record an ectopic pacemaker and then emit an ultrasound wave (7) through an ultrasound probe (2), the ultrasound wave passes through the ablation electrode (5) and reaches a tissue (6), and then a reflected ultrasound wave (9) is formed and reaches the ultrasound probe for imaging. At the same time, the ablation electrode (5) conducts a radiofrequency current (8, dotted arrow) into the tissue (6), forming tissue damage. During the ablation, cold saline is infused through the cold saline infusion holes 3 to prevent formation of aschar at a contact interface between the catheter and the tissue, thereby reducing the resistance.

### DESCRIPTION OF EMBODIMENTS

The present disclosure is further described below with reference to the drawings and examples.

### EXAMPLE 1: A TWO-IN-ONE CATHETER FOR REAL-TIME ULTRASOUND MONITORING AND RADIOFREQUENCY ABLATION

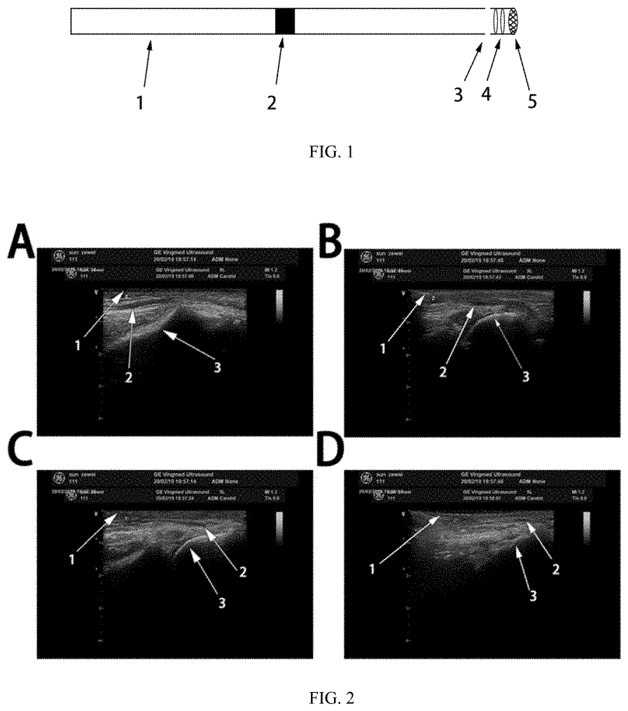

As shown in FIG. 1, the two-in-one catheter for real-time ultrasound monitoring and radiofrequency ablation is composed of a tube body 1, an ultrasound probe 2, cold saline infusion holes 3, recording electrodes 4, and an ablation electrode 5. The ultrasound probe 2 is located in a tube cavity of the tube body 1, the ablation electrode 5 is located at a head end of the tube body 1, the cold saline infusion holes 3 and the recording electrodes 4 are located at a front segment of the tube body 1. The ablation electrode 5 is made of a metal mesh or a metal column, in which a diameter of a metal wire forming the metal mesh or the metal column or a diameter of an aperture formed by the metal mesh or the metal column is adapted to the same order of magnitude of the wavelength of the ultrasound wave. An intraluminal ultrasound frequency commonly used in clinical practice is 5-15M, and thus the diameter of the metal wires forming the metal mesh or the metal column, or the diameter of the apertures formed by the metal mesh or the metal column is set to be 0.05 mm to 0.3 mm, such that the ultrasound wave can reach the back of the ablation electrode by diffraction for imaging. The cold saline perfusion holes 3 has a pore-like structure and include six cold saline perfusion holes 3 provided around the tube body, and they are configured to infuse cold saline during the ablation to prevent formation of eschar at a contact interface between the catheter and the tissue and reduce the resistance. The recording electrodes 4 are a pair of ring electrodes made of a platinum material and are fixed on the surface of the tube body 1. The recording electrodes 4 are fixed on the tube body in the same manner as a recording electrode on the ablation catheter (NS7TCDL174HS, Johnson & Johnson), and are configured to record an electrocardiogram of a portion of the myocardial tissue in contact with the catheter, so as to find an ectopic pacemaker of arrhythmia.

The ablation electrode 5 is preferably a metal mesh, and may be made of any metal, such as platinum, titanium, copper, iron, stainless steel, etc., preferably platinum or titanium.

### EXAMPLE 2: IMAGING USING AN ULTRASOUND PROBE WRAPPED WITH A METAL MESH

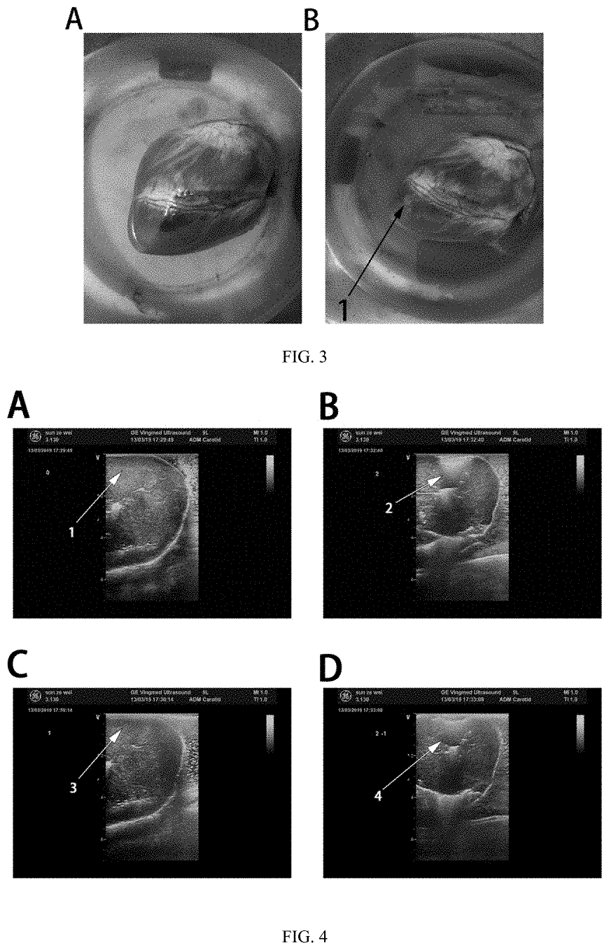

A 30-mesh copper mesh (a diameter of a copper wire is about 0.3 mm), a 100-mesh copper mesh (a diameter of a copper wire is about 0.1 mm), a 200-mesh copper mesh (a diameter of copper wire is about 0.05 mm) were respectively used to wrap the ultrasound probes, and then ultrasound imaging was performed at the inventor's wrist with an ultrasound frequency of 10M Hz. As shown in FIG. 2, FIG. 2A is an ultrasound image without a copper mesh in front of the ultrasound probe, where the arrow 1 denotes the skin, the arrow 2 denotes the blood vessel, and the arrow 3 denotes the bone. FIG. 2B is an ultrasound image with the 200-mesh copper mesh (the diameter of the copper wire is about 0.05 mm) wrapped around the ultrasound probe, FIG. 2C is an ultrasound image with the 100-mesh copper mesh (the diameter of the copper wire is about 0.1 mm) wrapped around the ultrasound probe, and FIG. 2D is an ultrasound image with the 30-mesh copper mesh (the diameter of the copper wire is about 0.3 mm) wrapped around the ultrasound probe, where the arrow 1 denotes the skin, the arrow 2 denotes the blood vessel, and the arrow 3 denotes the bone. Through comparison of FIGS. 2A, 2B, 2C, and 2D, it is obvious that the use of the metal mesh wrapping the ultrasound probe has no significant influence on the quality of ultrasound imaging.

### EXAMPLE 3: RADIOFREQUENCY ABLATION USING A METAL MESH AS AN ABLATION ELECTRODE

A metal mesh was fixed on a tip of disposable chopsticks, and the metal mesh was connected to a commercial ablation electrode with crocodile forceps. An isolated pig heart was placed in a basin, and physiological saline was placed in the basin. The metal mesh was attached on the surface of the pig heart, the back of the electrode was immersed in the saline in the basin, and then radiofrequency ablation was performed. FIG. 3A is an image before the radio frequency ablation, and FIG. 3B is an image after the radio frequency ablation using a metal mesh as an ablation electrode. It can be seen that tissue damage occurred in the ablation site and the color turned white (arrow 1).

### EXAMPLE 4: RESULTS OF ABLATION SUING A METAL MSH TO WRAP AN ULTRASOUND PROBE WHILE USING ANOTHER METAL MESH AS AN ABLATION ELECTRODE

A metal mesh was used to wrap an ultrasound probe, a pig heart was placed in a basin, saline was placed in the basin, and ultrasound imaging was performed before ablation. Subsequently, a metal mesh was fixed to the tip of disposable chopsticks, and the metal mesh was connected to a commercial ablation electrode with crocodile forceps. An isolated pig heart was placed in a basin, physiological saline was placed in the basin, the metal mesh was attached on the surface of the pig heart, the back of the electrode was immersed in the saline in the basin, and then radiofrequency ablation was performed. After the radiofrequency ablation, ultrasound imaging was performed again with the ultrasound probe wrapped with the metal mesh. FIG. 4A is an ultrasound image of a pig heart obtained by imaging with a simple ultrasound probe before the radiofrequency ablation, in which the arrow 1 denotes a left ventricle. FIG. 4B is an ultrasound image of a pig heart obtained by imaging with a simple ultrasound probe after the ablation using a metal mesh as an ablation electrode, and it can be seen that tissue damage occurred at the ablation site after the ablation (arrow 2). FIG. 4C is an ultrasound image of a pig heart obtained by imaging with a metal mesh-wrapped ultrasound probe before the radiofrequency ablation, where the arrow 3 denotes a left ventricle. It can be seen that the use of the metal mesh-wrapped ultrasound probe has no significant influence on the quality of ultrasound imaging. FIG. 4D is an ultrasound image obtained by imaging with a metal mesh-wrapped ultrasound probe after the radiofrequency ablation by using a metal mesh as an ablation electrode. It can be seen that ablation by using the metal mesh caused tissue damage, and the use of a metal mesh to wrap the ultrasound probe did not influence the quality of ultrasound imaging, and tissue damage (arrow 4) was observed after the ablation.

### EXAMPLE 5: APPLICATION OF THE ULTRASOUND AND RADIO FREQUENCY ABLATION TWO-IN-ONE CATHETER OF THE PRESENT DISCLOSURE

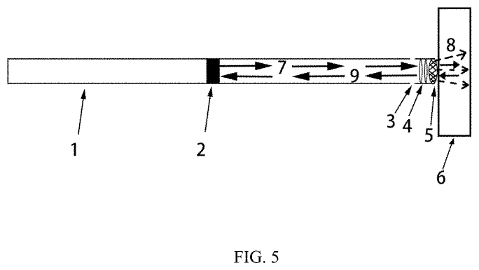

As shown in FIG. 5, a specific application method of the present disclosure is as follows: after an ectopic pacemaker is detected by the recording electrodes 4, an ultrasound wave is emitted from the ultrasound probe 2, the ultrasound wave passes through the ablation electrode 5 and reaches the tissue (6), and a reflected ultrasound wave 9 is formed and reaches the ultrasound probe for imaging. At the same time, the ablation electrode 5 conducts a radiofrequency current 8 (shown by a dashed arrow) into the tissue (6), forming tissue damage. The tissue damage is monitored in real time through ultrasound images. During ablation, cold saline is infused through the cold saline infusion holes 3 to prevent formation of eschar at the contact interface between the catheter and the tissue and reduce resistance.

It can be seen from the above examples that the ultrasound imaging will not be affected by placing a metal mesh in front of the ultrasound probe. Besides, the use of the metal mesh for radiofrequency ablation does not affect the ablation effect and can form the tissue damage. Therefore, by placing the ultrasound probe inside the catheter and using the metal mesh as the ablation electrode at the tip of the catheter, the present disclosure can achieve the following effects: the ultrasound can penetrate through the metal mesh for imaging, and in the meantime, the metal mesh is used as an ablation electrode for radiofrequency ablation, such that the radiofrequency ablation and the real-time ultrasound monitoring can be performed at the same time, thereby accurately reaching the predetermined tissue damage range. When the two-in-one catheter of the present disclosure is used for radiofrequency ablation, it is unnecessary to exchange the catheters, which is time-saving and reduces the difficulty of finding the ablation site after the catheters are exchanged. The radiofrequency ablation under the real-time ultrasound monitoring can also help the surgeon accurately reach the predetermined tissue damage range, thus the success rate of surgery is increased and the surgical complications are reduced. Therefore, the present disclosure is extremely helpful for the radiofrequency ablation surgery, is simple and easy to be implemented, and thus has the prospects of application.

What is claimed is:

1. A two-in-one catheter for real-time ultrasound monitoring and radiofrequency ablation, composed of a tube body (1), an ultrasound probe (2), cold saline perfusion holes (3), recording electrodes (4), and an ablation electrode (5), wherein the ultrasound probe (2) is located in a tube cavity of the tube body (1), the ablation electrode (5) is located at a head end of the tube body (1), and the cold saline perfusion holes (3) and the recording electrodes (4) are located in a front segment of the tube body (1).

2. The two-in-one catheter for real-time ultrasound monitoring and radiofrequency ablation according to claim 1, wherein the cold saline perfusion holes (3) have a pore-like structure, and include six cold saline perfusion holes (3) provided around the tube body (1), and the recording electrodes (4) are a pair of ring electrodes made of a platinum material and are fixed on a surface of the tube body (1).

3. The two-in-one catheter for real-time ultrasound monitoring and radiofrequency ablation according to claim 1, wherein the ablation electrode (5) is made of a metal mesh or a metal column, and a diameter of a metal wire forming the metal mesh or the metal column or a diameter of an aperture formed by the metal mesh or the metal column is adapted to the same order of magnitude of the wavelength of the ultrasound wave.

4. The two-in-one catheter for real-time ultrasound monitoring and radiofrequency ablation according to claim 2, wherein a diameter of a metal wire forming the metal mesh or the metal column or a diameter of an aperture formed by the metal mesh or the metal column is set to be in a range of 0.05 mm to 0.3 mm.

5. The two-in-one catheter for real-time ultrasound monitoring and radiofrequency ablation according to claim 1, wherein a material of the ablation electrode (5) is platinum, titanium, copper, iron, or stainless steel.
